(19) United States
(12) Patent Application Publication
(10) Pub. No.: US 2025/0288278 A1
(43) Pub. Date: Sep. 18, 2025

(54) JELLY BOTTLE HANGER

(71) Applicant: FUJIFILM Corporation, Tokyo (JP)

(72) Inventor: Masato EDA, Tokyo (JP)

(73) Assignee: FUJIFILM Corporation, Tokyo (JP)

(21) Appl. No.: 19/077,018

(22) Filed: Mar. 11, 2025

(30) Foreign Application Priority Data
Mar. 18, 2024 (JP) 2024-042695

(51) Int. Cl. A61B 8/00

(52) U.S. Cl. CPC A61B 8/4281; A61B 8/4411

(57) ABSTRACT

A jelly bottle hanger includes a winding portion that is wound around an ultrasound jelly bottle, and a hanging portion that is connected to two positions at which both ends are separated from each other in a longitudinal direction of the winding portion. The winding portion and the hanging portion are integrally formed of an elastic material. The ultrasound jelly bottle is held by tightening the ultrasound jelly bottle by the winding portion using elasticity. The ultrasound jelly bottle is suspended and supported by hooking the hanging portion on any hook.

### CROSS-REFERENCE TO RELATED APPLICATION

This application claims the priority benefit of Japan application serial no. 2024-042695, filed on Mar. 18, 2024. The entirety of the above-mentioned patent application is hereby incorporated by reference herein and made a part of this specification.

### BACKGROUND OF THE INVENTION

### 1. Field of the Invention

The present invention relates to a jelly bottle hanger that suspends and supports an ultrasound jelly bottle.

### 2. Description of the Related Art

In ultrasound diagnosis, in order to relieve a sharp change in impedance between a body of a patient and an ultrasound probe, an ultrasound jelly may be applied to a body surface of the patient. The ultrasound jelly is accommodated in a bottle formed of a flexible material and is discharged from the bottle by pressing the bottle. The ultrasound diagnostic apparatus may include a holder that holds the ultrasound jelly bottle, and the holder holds the bottle with the mouth down, so that the ultrasound jelly is discharged immediately after the bottle is taken out.

JP1999-227779A (JP-H11-227779A) and JP2009-285331A disclose an instrument that suspends a bottle accommodating a medical liquid with a discharge port downward.

### SUMMARY OF THE INVENTION

There is a case where the ultrasound jelly bottle is desired to be placed at a location other than the holder provided in the ultrasound diagnostic apparatus in a state where the discharge port is directed downward. In addition, the shape and size of the ultrasound jelly bottle vary, and it is desirable for the bottle that holds the ultrasound jelly to be able to accommodate the varying shapes and sizes.

An object of the present invention is to provide a technique for holding an ultrasound jelly bottle in a state in which a discharge port is directed downward at a place other than a holder of an ultrasound diagnostic apparatus.

A jelly bottle hanger of the present invention comprises a winding portion that is wound around the ultrasound jelly bottle, and a hanging portion that is connected to two positions at which both ends are separated from each other in a longitudinal direction of the winding portion, in which the winding portion and the hanging portion are integrally formed of an elastic material. The ultrasound jelly bottle is held by being tightened by the winding portion.

The winding portion formed of an elastic material stretches, so that the winding portion can correspond to ultrasound jelly bottles having different diameters.

The winding portion may include a long belt portion, and a hook ring portion that is joined to one end of the belt portion and in which a through hole through which the belt portion passes is formed. The belt portion may include a hook protrusion, and at least one hook hole that is hooked on the hook protrusion may be formed in a portion of the belt portion that is folded back through the through hole. The belt portion is folded back through the through hole, and the hook hole is hooked on the hook protrusion, whereby the ultrasound jelly bottle can be reliably tightened and held.

A plurality of the hook holes may be formed, and the plurality of hook holes are arranged along a longitudinal direction of the belt portion. By selecting the hook hole to be used, it is possible to correspond to ultrasound jelly bottles having different diameters.

A thickness of the belt portion may be 2 to 3 mm. The step formed on the side surface of the ultrasound jelly bottle comes into contact with the hand gripping the bottle depending on the thickness of the belt portion, and the bottle is reliably held.

A thickness of the hook ring portion may be larger than the thickness of the belt portion. The belt portion is likely to be passed through the through hole of the hook ring portion.

By forming the jelly bottle hanger with an elastic material, the jelly bottle hanger can correspond to the ultrasound jelly bottles having different diameters by extending and contracting the belt portion wound around the ultrasound jelly bottle.

### BRIEF DESCRIPTION OF THE DRAWINGS

FIG. 1 is a perspective view showing a schematic configuration of an ultrasound diagnostic apparatus.

FIGS. 2A to 2C are views showing a jelly bottle hanger in a state of being attached on an ultrasound jelly bottle.

FIGS. 3A to 3C are views showing a jelly bottle hanger alone.

### DESCRIPTION OF THE PREFERRED EMBODIMENTS

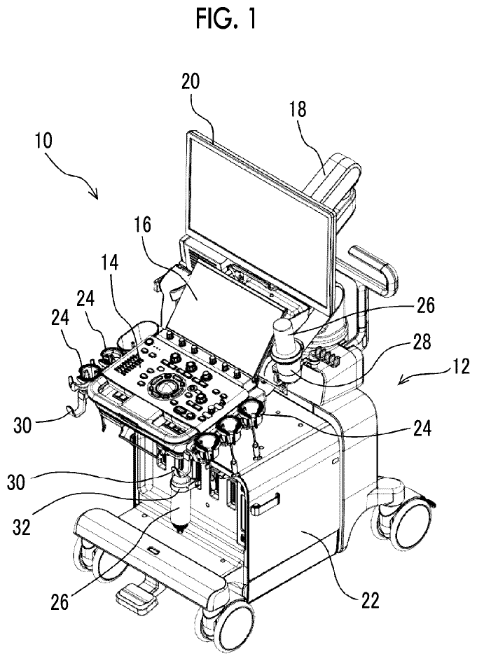

Hereinafter, embodiments of the present invention will be described with reference to the drawings. FIG. 1 is a perspective view showing an appearance of an ultrasound diagnostic apparatus 10. The ultrasound diagnostic apparatus 10 includes an apparatus main body 12, an operation panel 14 and a sub monitor 16 supported above the apparatus main body 12, and a main monitor 20 supported by a multi-joint arm 18 mounted on the apparatus main body 12. The apparatus main body 12 has a box-shaped accommodation unit 22 that accommodates a control circuit that controls transmission and reception of ultrasound waves, a power supply circuit, a connector to which the ultrasound probe is connected, and the like. The ultrasound image is displayed on the main monitor 20. A setting condition and the like during diagnosis are displayed on the sub monitor 16. On the operation panel 14, operators such as push buttons, levers, and track balls for performing operations such as setting various parameters in diagnosis and operating the ultrasound probe are disposed. A probe holder 24 that holds a head of the ultrasound probe is disposed on the left and right sides of the operation panel 14. A plurality of probe holders 24 may be disposed. Further, a jelly bottle holder 28 that holds the ultrasound jelly bottle 26 is provided in the vicinity of the operation panel 14. Hooks 30 are provided on left and right sides of a front edge of the operation panel 14. The hook 30 can hook a cord of the ultrasound probe or the like, and in FIG. 1, the ultrasound jelly bottle 26 on which the jelly bottle hanger 32 is attached is hooked thereon.

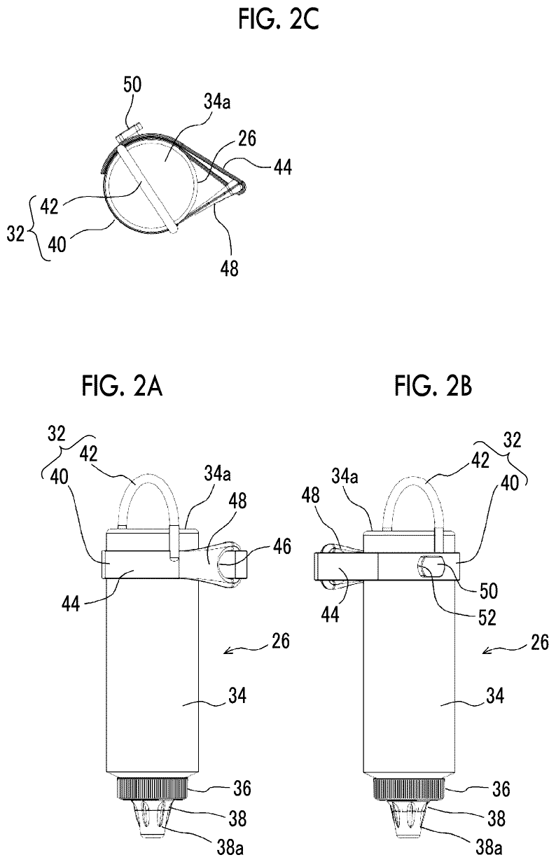

FIGS. 2A to 2C are views showing the ultrasound jelly bottle 26 on which the jelly bottle hanger 32 is attached. FIG. 2A is a front view, FIG. 2B is a rear view, and FIG. 2C is a plan view. The ultrasound jelly bottle 26 includes a cylindrical bottle main body 34 and a bottle cap 36 that is screw-coupled to one end of the bottle main body 34. The bottle cap 36 has a discharge nozzle 38 that discharges an ultrasound jelly. When the tip part 38a of the discharge nozzle 38 is rotated, the discharge port for discharging the ultrasound jelly opens at the tip.

The jelly bottle hanger 32 is attached on an end portion of the bottle main body 34 on the bottom surface 34a side. The jelly bottle hanger 32 has a winding portion 40 that is wound around the bottle main body 34, and a hanging portion 42 of which both ends are respectively connected to two positions of the winding portion 40. The hanging portion 42 is hooked on a hook 30 of the ultrasound diagnostic apparatus 10, a hook provided on another device, or the like. As a result, the ultrasound jelly bottle 26 is suspended with the discharge nozzle 38 downward, that is, in an inverted state. In a state where the ultrasound jelly bottle 26 is suspended, the hanging portion 42 of the jelly bottle hanger 32 crosses above the bottom surface 34a of the bottle main body 34 while being curved vertically.

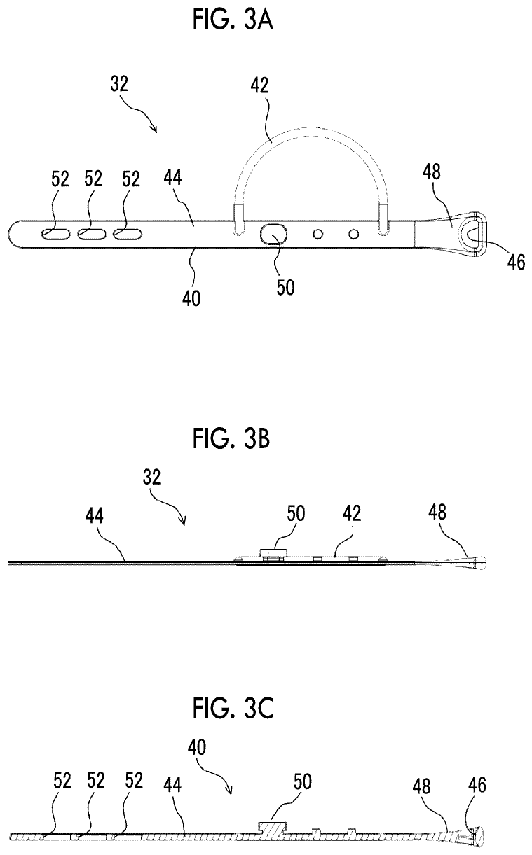

FIGS. 3A to 3C are views showing the jelly bottle hanger 32 alone. FIG. 3A is a plan view, FIG. 3B is a front view, and FIG. 3C is a cross-sectional view of the winding portion 40. The dimension in the thickness direction is enlarged in the cross-sectional view.

The jelly bottle hanger 32 has a winding portion 40 that extends straight when the ultrasound jelly bottle 26 is not attached, and a hanging portion 42 of which both ends are respectively connected to two positions separated from each other in the longitudinal direction of the winding portion 40. The jelly bottle hanger 32 is integrally formed of an elastic material such as rubber. The winding portion 40 has a long belt portion 44 that occupies most of the length of the winding portion 40, and a hook ring portion 48 that is joined to one end (right end in FIGS. 3A to 3C) of the belt portion 44 and in which a through hole 46 for passing the belt portion 44 is provided.

A hook protrusion 50 is provided at a substantially center portion of the belt portion 44 in the longitudinal direction. The hook protrusion 50 may be provided between the two connecting points of the hanging portion 42. The hook protrusion 50 may be a mushroom-shaped protrusion in which a root part is thin and a tip part is thick. A hook hole 52 is formed at an end part (a left end part in FIGS. 3A to 3C) of the belt portion 44 on a side opposite to the hook ring portion 48 so as to penetrate the belt portion 44. The hook hole 52 may be a long hole that is long in the longitudinal direction of the belt portion 44. Three hook holes 52 are arranged along the longitudinal direction of the belt portion 44. The number of the hook holes 52 may be other than three, and may be one.

The thickness of the belt portion 44 may be 2 to 3 mm, and is 2 mm in the jelly bottle hanger 32. The belt portion 44 having a thickness of about 2 mm is wound around the ultrasound jelly bottle 26, whereby a step is formed on the side surface of the bottle main body 34. In a case of handling the ultrasound jelly bottle 26, the step is hooked on the hand gripping the ultrasound jelly bottle 26, and the ultrasound jelly bottle 26 is reliably held. The thickness of the hook ring portion 48 may be thicker than that of the belt portion 44. The thickness of the winding portion 40 may have a step at the boundary between the belt portion 44 and the hook ring portion 48, or may change smoothly.

A method of attaching the jelly bottle hanger 32 on the ultrasound jelly bottle 26 will be described with reference to FIGS. 2A to 2C. The belt portion 44 of the jelly bottle hanger 32 is wound around the periphery of the bottle main body 34 of the ultrasound jelly bottle 26 such that the hook protrusion 50 is on the outside. The belt portion 44 is wound around an end portion of the bottle main body 34 on the bottom surface 34a side. In this case, the hook ring portion 48 is separated from the bottle main body 34. A tip of the belt portion 44, that is, an end opposite to a side to which the hook ring portion 48 is connected, is passed through the through hole 46 of the hook ring portion 48. In this case, the tip of the belt portion 44 is passed from the side (inside) close to the bottle main body 34 to the side (outside) far from the bottle main body 34. A portion of the belt portion 44 that has passed through the through hole 46 is folded back and is superimposed on a portion of the bottle main body 34 that is wound. Further, any of the hook holes 52 is hooked on the hook protrusion 50. The winding portion 40 is extending and is curved along the bottle main body 34 in a case of being attaching on the ultrasound jelly bottle. The winding portion 40 tightens the bottle main body 34 with a force with which the extending winding portion 40 is to be shrunk, and the jelly bottle hanger 32 holds the ultrasound jelly bottle 26.

The jelly bottle hanger 32 can correspond to ultrasound jelly bottles having different diameters by extending and contracting the belt portion 44. In addition, for the ultrasound jelly bottle having a larger diameter difference, the corresponding response can be made by changing the hook hole 52 to be hooked on the hook protrusion 50.

In FIGS. 2A to 2C, the hook ring portion 48 is separated from the side surface of the bottle main body 34 due to a difference in stiffness from the belt portion 44. The hook ring portion 48 may be curved instead of extending straight as shown in FIGS. 2A to 2C. Instead of this, the hook ring portion 48 may also be configured to be curved along the side surface of the bottle main body 34.

What is claimed is:

1. A jelly bottle hanger for suspending an ultrasound jelly bottle, comprising:
a winding portion that is wound around the ultrasound jelly bottle; and
a hanging portion that is connected to two positions at which both ends are separated from each other in a longitudinal direction of the winding portion,
wherein the winding portion and the hanging portion are integrally formed of an elastic material, and
the ultrasound jelly bottle is held by being tightened by the winding portion.

2. The jelly bottle hanger according to claim 1,
wherein the winding portion includes a long belt portion and a hook ring portion that is joined to one end of the belt portion and in which a through hole through which the belt portion passes is formed,
the belt portion includes a hook protrusion, and
at least one hook hole that is hooked on the hook protrusion is formed in a portion of the belt portion that is folded back through the through hole.

3. The jelly bottle hanger according to claim 2,
wherein a plurality of the hook holes are formed, and
the plurality of hook holes are arranged along a longitudinal direction of the belt portion.

4. The jelly bottle hanger according to claim 2,
wherein a thickness of the belt portion is 2 to 3 mm.

5. The jelly bottle hanger according to claim 4,
wherein a thickness of the hook ring portion is larger than the thickness of the belt portion.
